(19) United States
(12) Patent Application Publication
(10) Pub. No.: US 2008/0243090 A1
(43) Pub. Date: Oct. 2, 2008

(54) Nozzle assembly for enema kit

(76) Inventor: Lung Sheng Tsai, Taoyuan City (TW)

Correspondence Address: RABIN & Berdo, PC, 1101 14TH STREET, NW, SUITE 500, WASHINGTON, DC, 20005, US

(21) Appl. No.: 11/727,899

(22) Filed: Mar. 28, 2007

(51) Int. Cl. A61M 5/00

(52) U.S. Cl. 604/261

(57) ABSTRACT

A nozzle assembly for enema kit includes an extension tube curved in a predetermined manner, and a plug body connected at a proximal end to a first end of the extension tube. The plug body has a distal end formed into a nozzle, which has a predetermined short length and is provided at a front end with a plurality of nozzle apertures, and includes a rearward flared cup portion. When a user uses the enema kit to do colonic hydrotherapy with the nozzle introduced through the user's anal sphincter, water is supplied to the nozzle assembly and jetted into the user's anus. A rearward flared cup portion is formed behind the nozzle to press against the user's butt, preventing the nozzle from moving too deep into the user's anus, making the enema kit comfortable and safe for use by a non professional person.

### FIELD OF THE INVENTION

The present invention relates to a nozzle assembly for enema kit, and more particularly to a nozzle assembly movably connected to a water conduit on a locating board of an enema kit for supplying water during a colonic hydrotherapy.

### BACKGROUND OF THE INVENTION

A human's large intestine generally has a shape of a reverse letter U, and includes a length of descending colon located above the rectum, a length of transverse colon extended from an upper end of the descending colon, and a length of ascending colon downward extended from the other end of the transverse colon.

Dr. Bernard Jensen of John Hopkins Medical College conducted a study based on 50 thousands cases and developed a Map of Point Locations in Large Intestine. According to Dr. Bernard Jensen, excrements accumulated at any points in the large intestine indicate some particular areas of a person are in an abnormal condition. For example, statistic data from the study indicates excrements accumulated at a point in the ascending colon denoted by numeral 8 in the map indicates a person has abnormality in thyroid gland or pituitary gland; and excrements accumulated at points in the transverse colon denoted by numerals 63, 64 in the map indicate a person has abnormalities at esophagus or bronchial tubes. Since the human large intestine has complicate structure and turns, the only way to cleanse the excrements accumulated at the joints of the transverse colon and the descending colon and the ascending colon is to conduct colonic hydrotherapy.

There are home enema kits commercially available in the market for non-professional users to do enema at home. Most conventional home enema kits include an upright plug or a cleansing tube in contact with the user's anus. Such upright plug will cause discomfort to the user, making the user fearful of enema. For non-professional users, injury to the large intestine caused by home enema kit often occurs due to poor handling skill thereof. This is also a factor prevents users from trying the home enema kits.

Moreover, the plug for the conventional home enema kits is relatively long to cause discomfort even pain to the user's anal sphincter during the 10 to 20 minutes long process of colonic hydrotherapy.

Therefore, while the conventional home enema kits allow people to conduct enema at home without the help of a professional nursing person, they still require improvements to ensure the users' safety and comfortableness in using the enema kits.

### SUMMARY OF THE INVENTION

A primary object of the present invention is to provide a nozzle assembly for enema kit, so that a user may conveniently conduct a colonic hydrotherapy by introducing the nozzle assembly through the user's anal sphincter.

Another object of the present invention is to provide a nozzle assembly for enema kit, which includes a shortened nozzle forward extended from a plug body, and a rearward flared cup portion directly behind the nozzle. The shortened nozzle does not cause discomfort to the user when it is introduced into the user's anus, and the flared cup portion presses against the user's butt to safely prevent the nozzle from moving too deep into the user's anus.

A further object of the present invention is to provide a disposable nozzle assembly for movably connecting to a water conduit of an enema kit to jet water into a user's anus, so that the nozzle assembly can be disassembled from the enema kit and discarded after it has been used, and thereby meets the requirements of personal sanitation and environmental protection.

To achieve the above and other objects, the nozzle assembly for enema kit according to the present invention includes an extension tube in a predetermined curved shape, and a plug body connected at a proximal end to a first end of the extension tube. The plug body has a distal end formed into a nozzle, which has a predetermined short length and is provided at a front end with a plurality of nozzle apertures, and includes a rearward flared cup portion.

In a preferred embodiment of the present invention, the extension tube is connected at an opposite second end to a water conduit on a locating board of the enema kit.

In an ideal embodiment of the present invention, the plug body further includes an interconnecting section for connecting to the first end of the extension tube.

In an operable embodiment of the present invention, the extension tube is an integral part of the plug body to extend therefrom.

To use the nozzle assembly of the present invention, first connect the first end of the extension tube to the interconnecting section of the plug body, and then connect the second end of the extension tube to the water conduit on the locating board of the enema kit.

To use the enema kit to do colonic hydrotherapy, first position a main unit of the kit on a toilet tank lid, and the locating board across a rear rim of the toilet bowl, and then turn a toilet seat downward to clamp the locating board between the toilet seat and the bowl. The user sits on the toilet seat with the nozzle introduced into the user's anus, and controls the main unit to supply water to the nozzle via the water conduit, so that water is jetted from the nozzle into the user's anus for cleansing colon.

The shortened nozzle does not cause discomfort to the user, helping a user to overcome the fear of enema; and the rearward flared cup portion formed behind the nozzle presses against the user's butt, preventing the nozzle from moving too deep into the user's anus, making the enema kit comfortable and safe for use by a non professional person.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an exploded perspective view of a nozzle assembly for enema kit according to an embodiment of the present invention;

FIGS. 2 and 3 show the manner of connecting the nozzle assembly of the present invention to a locating board included in the enema kit;

FIGS. 4 and 5 show a complete enema kit with the nozzle assembly of the present invention assembled thereto for use with a toilet; and

FIG. 6 shows the nozzle assembly of the present invention introduced through a user's anal sphincter for colonic hydrotherapy.

### DETAILED DESCRIPTION OF THE PREFERRED EMBODIMENTS

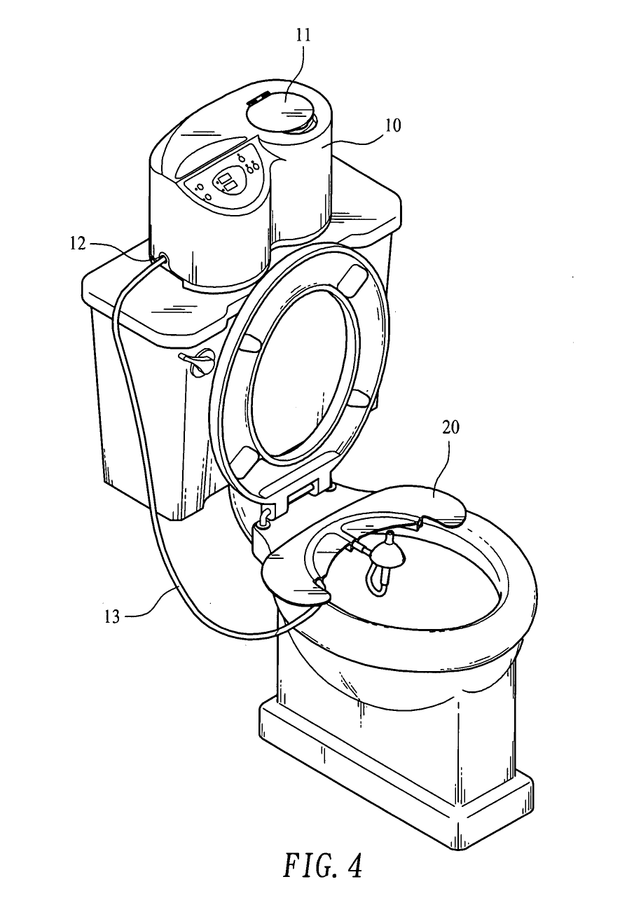

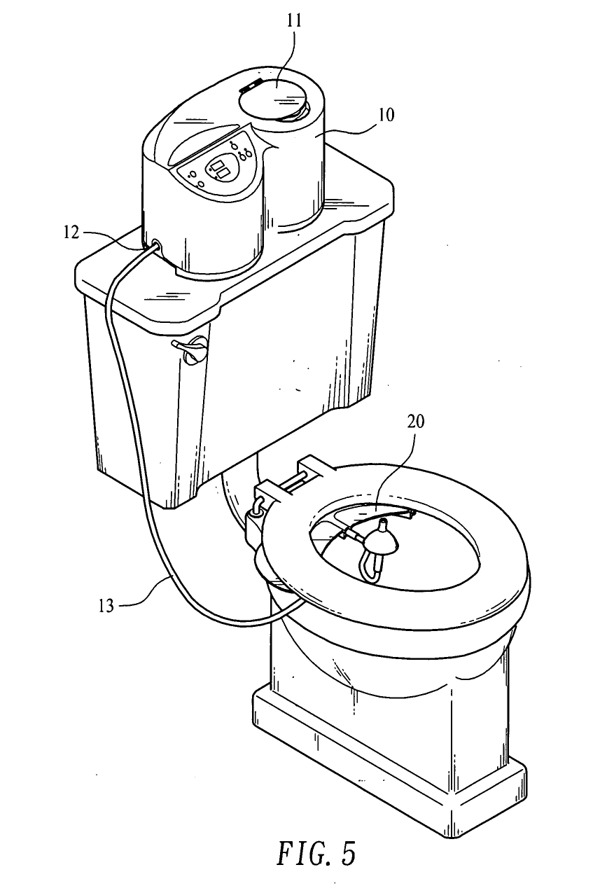

Please refer to FIGS. 4 and 5. A general enema kit for home colonic hydrotherapy includes a main unit 10 for positioning on a tank lid of a toilet. The main unit 10 is provided at one side with a water inlet 11, via which water needed for enema is supplied for heating and sterilizing. The main unit 10 may be controlled by a user to operate and display messages of working pressure, temperature, water flow, and water low, so that the user may always monitor the process of pressurizing and feeding water, and obtain information thereof. The main unit 10 is provided at another side with a water outlet 12, which is connected to a locating board 20 via a hose 13.

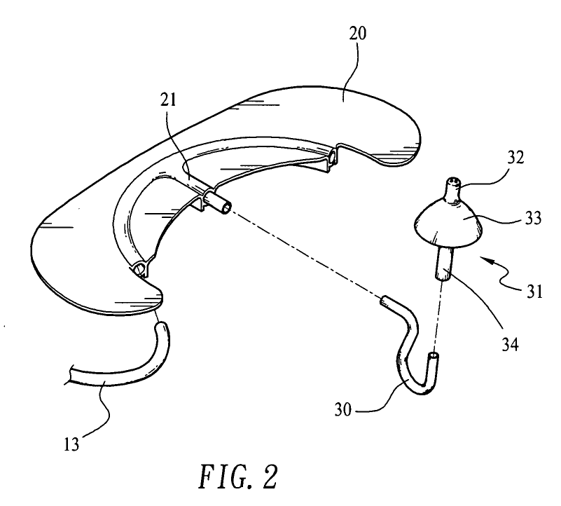

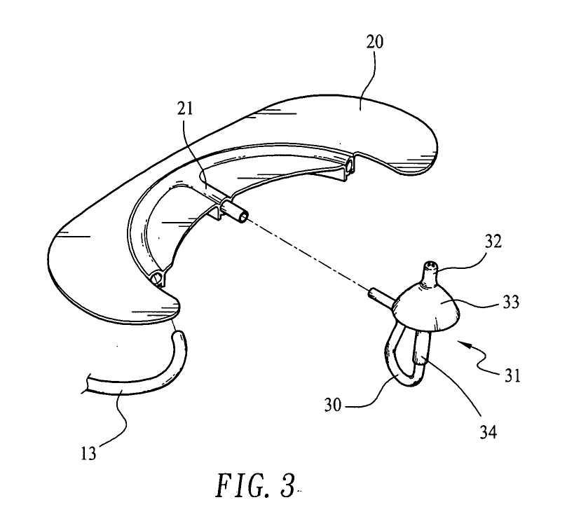

Please also refer to FIGS. 2 and 3. The locating board 20 has a configuration generally corresponding to a rear rim of a bowl of the toilet, so as to straddle the rear rim of the bowl. The locating board 20 is provided along an inner edge or an inner side with a preformed water conduit 21.

As can be seen from FIGS. 4 and 5, after the main unit 10 is positioned on the tank lid and the locating board 20 is located on the rear rim of the bowl, a toilet seat may be turned downward to clamp the locating board 20 between the seat and the bowl.

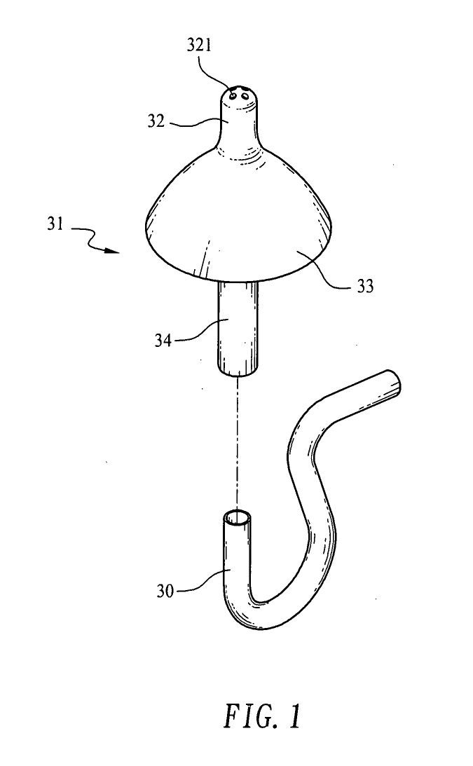

Please refer to FIG. 1 that is an exploded perspective view of a nozzle assembly for the above described enema kit; and refer to FIGS. 2 and 3 that show the manner of connecting the nozzle assembly to the locating board 20. As shown, the nozzle assembly includes an extension tube 30 curved in a predetermined manner, a first end of which is movably connected to a plug body 31. The plug body 31 has a distal end formed into a nozzle 32, which forward extends by a predetermined length; and a flared cup portion 33 rearward extended from the nozzle 32. Nozzle apertures 321 are formed at a front end of the nozzle 32.

In a preferred embodiment of the present invention, a second end of the extension tube 30 opposite to the first end is movably connected to the water conduit 21 on the locating board 20.

In an ideal embodiment of the present invention, the plug body 31 is further provided at the proximal end with an interconnecting section 34, to which the first end of the extension tube 30 is connected.

In an operable embodiment of the present invention, the extension tube 30 is an integral part of the plug body 31 to extend therefrom.

Before using the enema kit, the plug body 31 is connected at the interconnecting section 34 to the first end of the extension tube 30, and then, the extension tube 30 is movably connected at the second end to the water conduit 21 on the locating board 20.

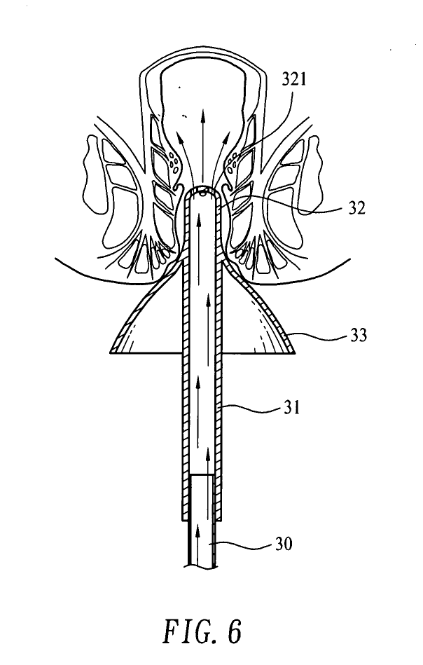

Please refer to FIG. 6. When a user conducts a colonic hydrotherapy using the enema kit shown in FIGS. 4 and 5, the user first sits on the toilet seat and introduces the nozzle 32 in front of the plug body 31 through the anal sphincter. Then, the user controls the main unit 10 for the previously prepared disinfected water to flow from the main unit 10 via the water conduit 21 on the locating board 20 and the extension tube 30 into the plug body 31, so that the water is jetted out of the nozzle 32 via the nozzle apertures 321 into the anus for colonic hydrotherapy. During the process of colonic hydrotherapy, the user may control the operation of the main unit 10 via a remote controller or touching a control panel on the main unit 10.

In the present invention, the nozzle 32 at the distal end the plug body 31 has a predetermined short length to prevent the user from discomfort during the colonic hydrotherapy. Moreover, the flared cup portion 33 rearward extended from the nozzle 32 is smoothly pressed against the user's butt when the nozzle 32 has been introduced into the user's anus, preventing the nozzle 32 from moving too deep into the anal sphincter. Therefore, the enema kit with the nozzle assembly of the present invention may be used without the assistance of a professional nursing person, and protects the user against any injury due to unskilled operation of the nozzle assembly by the user. Since the nozzle assembly for enema kit according to the present invention is comfortable and safe for use, it largely reduces the user's fear of enema.

The nozzle assembly of the present invention can be easily assembled to the locating board 20 to receive water supplied from the main unit 10 for colonic hydrotherapy, and can be easily disassembled from the locating board 20 and discarded after each colonic hydrotherapy to meet the requirements of personal sanitation and environmental protection. The present invention is therefore convenient and simple for use in enema.

The present invention has been described with a preferred embodiment thereof and it is understood that many changes and modifications in the described embodiment can be carried out without departing from the scope and the spirit of the invention that is intended to be limited only by the appended claims.

What is claimed is:

1. A nozzle assembly for enema kit, comprising an extension tube curved in a predetermined manner, and a plug body connected at a proximal end to a first end of the extension tube; the plug body having a distal end formed into a nozzle, which has a predetermined length and is provided at a front end with a plurality of nozzle apertures, and includes a rearward flared cup portion.

2. The nozzle assembly for enema kit as claimed in claim 1, wherein the extension tube is movably connected at a second end opposite to the first end to a water conduit provided on a locating board included in the enema kit.

3. The nozzle assembly for enema kit as claimed in claim 1, wherein the plug body further includes an interconnecting section rearward extended from the nozzle for movably connecting to the first end of the extension tube.

4. A nozzle assembly for enema kit, comprising an extension tube which is curved in a predetermined manner and has a first end integrally extended from a proximal end of a plug body; and the plug body having a distal end formed into a nozzle, which has a predetermined length and is provided at a front end with a plurality of nozzle apertures, and includes a rearward flared cup portion.

5. The nozzle assembly for enema kit as claimed in claim 4, wherein the extension tube is movably connected at a second end opposite to the first end to a water conduit provided on a locating board included in the enema kit.
